(19) United States
(12) Patent Application Publication
(10) Pub. No.: US 2022/0324459 A1
(43) Pub. Date: Oct. 13, 2022

(54) DETERMINATION DEVICE AND RECORDING MEDIUM

(71) Applicant: KABUSHIKI KAISHA TOKAI RIKA DENKI SEISAKUSHO, Aichi (JP)

(72) Inventors: Kenji NARUMI, Aichi (JP); Mitsuo MORI, Aichi (JP)

(73) Assignee: KABUSHIKI KAISHA TOKAI RIKA DENKI SEISAKUSHO, Aichi (JP)

(21) Appl. No.: 17/623,958

(22) PCT Filed: Mar. 27, 2020
(86) PCT No.: PCT/JP2020/014059

§ 371 Date: Dec. 30, 2021

(30) Foreign Application Priority Data
Jul. 12, 2019 (JP) 2019-130302

(51) Int. Cl. B60W 40/08; A61B 3/113; A61B 5/11; A61B 5/00; A61B 5/18; G08B 21/06

(52) U.S. Cl. CPC B60W 40/08; A61B 3/113; A61B 5/1122; A61B 5/4809; A61B 5/18; G08B 21/06; B60W 2040/0872

(57) ABSTRACT

To provide a determination device and program that make it possible to improve accuracy of determining a sign related to a decrease in driving ability of a driver.

Provided is a determination device including: a sign determination section configured to determine a sign related to a decrease in driving ability of a driver who drives a mobile object on a basis of head movement measurement data obtained by measuring head movement of the driver and eye movement measurement data obtained by measuring eye movement of the driver; and a head swing control section configured to control a head swing section in such a manner that the head swing section swings the head of the driver.

### TECHNICAL FIELD

The present invention relates to a determination device and a program.

### BACKGROUND ART

In recent years, technologies of detecting an arousal state of a driver and taking various kinds of measures while the driver is driving a vehicle or the like have been proposed. For example, Patent Literature 1 discloses a sleepiness sign detection apparatus configured to detect vestibulo-ocular reflex (VOR) from data obtained by measuring eye movement, head movement, or the like of a driver of a vehicle or an operator of a machine and determine a sign of sleepiness on a basis of the VOR.

### CITATION LIST

### Patent Literature

Patent Literature 1: JP 5255063 B2

### DISCLOSURE OF INVENTION

### Technical Problem

However, the technology disclosed in Patent Literature 1 has a problem that it is not possible to determine the sign of sleepiness on a basis of the VOR in the case where the head movement that is large enough to induce the VOR has not occurred spontaneously. Therefore, the technology disclosed in Patent Literature 1 has room for improvement in accuracy of determining a sign related to a decrease in driving ability of a driver such as sleepiness.

Accordingly, the present invention is made in view of the aforementioned problem, and an object of the present invention is to provide a novel and improved determination device and program that make it possible to improve the accuracy of determining a sign related to a decrease in driving ability of a driver.

### Solution to Problem

To solve the problem, according to an aspect of the present invention, there is provided that a determination device comprising, a sign determination section configured to determine a sign related to a decrease in driving ability of a driver who drives a mobile object on a basis of head movement measurement data obtained by measuring head movement of the driver and eye movement measurement data obtained by measuring eye movement of the driver, and a head swing control section configured to control a head swing section in such a manner that the head swing section swings the head of the driver.

The head swing control section may control the head swing section at a timing depending on at least a state of the mobile object or a state of the driver.

The timing depending on the state of the mobile object may be a timing when the mobile object is assumed to vibrate.

The timing depending on the state of the driver may be a timing when the driving ability of the driver is predicted to decrease.

The head swing control section may control the head swing section in a case where the head movement measurement data is smaller than a predetermined value.

The sign determination section may detect vestibulo-ocular reflex induced by the head movement on a basis of the head movement measurement data and the eye movement measurement data, and determines the sign related to the decrease in the driving ability of the driver on a basis of the vestibulo-ocular reflex, and the predetermined value may be a value of the head movement that makes it possible to induce the vestibulo-ocular reflex.

The head swing control section may control the head swing section in a case where head movement beyond the predetermined value has not been measured for a predetermined period of time.

The head swing control section may control the head swing section at a certain interval.

The head swing section may be a webbing winding device configured to wind webbing worn by the driver, an adjustment mechanism of a driver's seat, or a vibration section installed in the driver's seat.

The head swing control section may control at least any of the webbing winding device, the adjustment mechanism of the driver's seat, or the vibration section installed in the driver's seat.

To solve the problem, according to other aspect of the present invention, there is provided that a program that causes a computer to function as a sign determination section configured to determine a sign related to a decrease in driving ability of a driver who drives a mobile object on a basis of head movement measurement data obtained by measuring head movement of the driver and eye movement measurement data obtained by measuring eye movement of the driver and a head swing control section configured to control a head swing section in such a manner that the head swing section swings the head of the driver.

### Advantageous Effects of Invention

As described above, according to the present invention, it is possible to improve the accuracy of determining a sign related to a decrease in driving ability of a driver.

### BRIEF DESCRIPTION OF DRAWINGS

FIG. 1 is a diagram for describing a sign determination system according to an embodiment of the present invention.

FIG. 2 is a block diagram illustrating a functional configuration example of a device for implementing the sign determination system according to the embodiment of the present invention.

FIG. 3 is a diagram illustrating an example of measurement data of head movement according to the embodiment of the present invention.

FIG. 4 is a flowchart illustrating an example of a flow of a sign determination process performed by a determination device according to the embodiment of the present invention.

### MODE(S) FOR CARRYING OUT THE INVENTION

Hereinafter, referring to the appended drawings, a preferable embodiment of the present invention will be described in detail. Note that, in this specification and the appended drawings, structural elements that have substantially the same function and structure are denoted with the same reference signs, and repeated explanation of these structural elements will be omitted.

<1. Overview>

As described above, in recent years, the technologies of detecting an arousal state of a driver and taking various kinds of measures have been proposed. However, the technologies of detecting vestibulo-ocular reflex and determining a sleepiness sign have a problem that it is not possible to determine the sign of sleepiness or accuracy of determining the sign decreases in the case where head movement that is large enough to induce the vestibulo-ocular reflex has not occurred spontaneously. For example, it is assumed that the head is unlikely to move and the vestibulo-ocular reflex is unlikely to be induced in the case where a road surface is in good condition or in the case where shaking felt by passengers in a vehicle is suppressed as much as possible.

The embodiments of the present invention was conceived by focusing on the above-described points, and makes it possible to improve accuracy of determining a sign related to a decrease in driving ability of a driver by swinging the head of the driver.

Here, the “vestibulo-ocular reflex” will be described first. The “vestibulo-ocular reflex” is used for determining a sign related to a decrease in driving ability according to the present embodiment. Human eye movement includes voluntary movement and involuntary movement. The involuntary movement is induced by a visual or vestibular sensory stimulus. Examples of the involuntary movement include the vestibulo-ocular reflex (VOR) induced by head movement. The VOR is a reflex rotating eyeballs in an opposite direction from a head rotating direction on the basis of information related to the head rotation detected by semicircular canals in inner ears. Magnitude and timing of the VOR to a stimulus are also used to evaluate motion control ability of a subject, for example.

In addition, it is known that the VOR exhibits characteristics closely related to sleepiness. As described above, the VOR is a type of eye movement induced by head movement. It is known that speed of the VOR of a human gradually decreases (delays) in the case where human motion control ability is decreased by sleepiness, dosing of anesthetic medication, or the like in a normal arousal state.

Therefore, by using the method of determining the sign according to the present embodiment, it is possible to measure head movement and eye movement of a driver, detect the VOR on the basis of the measured head movement and eye movement, and determine a sign related to a decrease in driving ability of the driver depending on the speed of the VOR.

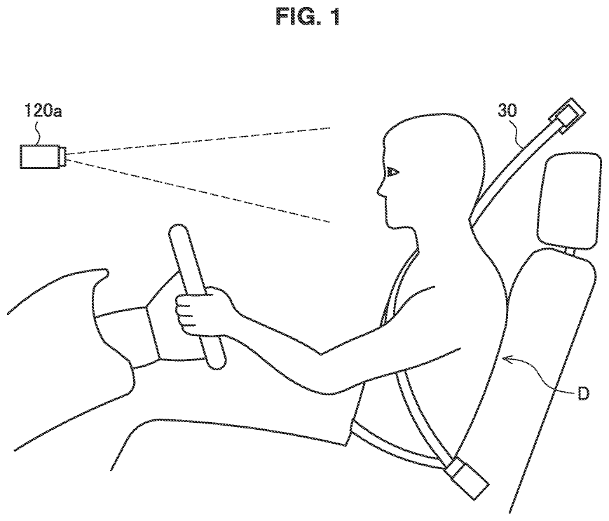

FIG. 1 is a diagram for describing a sign determination system according to an embodiment of the present invention. As illustrated in FIG. 1, an example of the mobile object according to the present embodiment is assumed to be a vehicle. For example, as illustrated in FIG. 1, a camera 120a disposed in the vehicle measures head movement and eye movement of a driver D who drives the vehicle. The camera 120a is capable of continuously capturing images of a face of the driver D and measuring the head movement and eye movement of the driver D on the basis of the moving image.

Note that, a measurement device (sensor section) configured to measure the head movement and eye movement of the driver D is not limited to the camera 120a. For example, the eye movement may be measured by an eye movement measurement section including a light emitting section and a light receptive section. The light emitting section is configured to emit near-infrared light. The light receptive section is configured to receive the near-infrared light. The light receptive section is capable of capturing images of the eyeballs of the driver D and measuring the eye movement by receiving near-infrared light that is emitted from the light emitting section and reflected by the eyeballs. For example, the light emitting section and the light receptive section may be disposed in a meter hood for a driver's seat or on an upper surface of a steering column. Such disposition makes it possible to measure the eye movement of the driver D in a non-contact manner.

In addition, the measurement of head movement and eye movement according to the present embodiment is not limited to the above-described non-contact measurement, but may be measurement performed in a contact manner. For example, it is possible to measure the head movement on the basis of sensing data obtained by a wearable device (such as a glasses-type device or a device to be attached to glasses) provided with a gyro sensor, an acceleration sensor, or the like in the case where the wearable device is worn on the head of the driver D. In addition, the present system is also capable of measuring the eye movement on the basis of data detected by an ocular potential sensor, an inward-facing camera (that faces a direction (inward direction) capable of capturing images of the eyes of the driver when the drives wears the wearable device), or the like installed in the wearable device.

Next, the present system detects the VOR on the basis of the measured head movement and eye movement, and determines the sign related to a decrease in driving ability of the driver such as a sleepiness sign.

Here, as described above, the VOR is not induced if the head does not move, and it is not possible to determine the sleepiness sign. Therefore, the present system is capable of improving accuracy of determining the sign related to a decrease in driving ability by moving webbing 30 or the like worn by the driver, swinging the head of the driver, inducing the VOR, and detecting the VOR more certainly. Note that, the way of swinging the head of the driver is not limited to the example of moving the webbing 30. Other specific examples will be described later.

<2. Configuration Example>

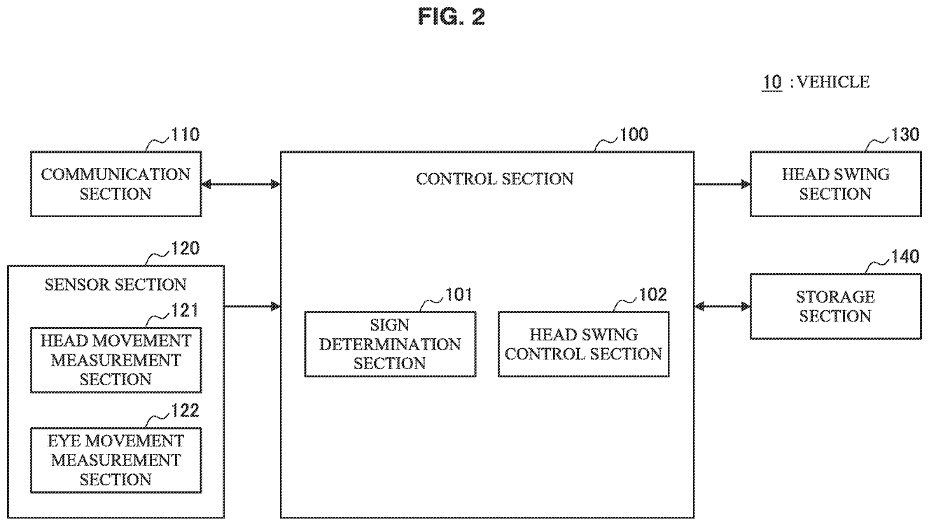

Next, a configuration example of a determination device for implementing the sign determination system according to the present embodiment will be described. FIG. 2 is a block diagram illustrating a functional configuration example of a device for implementing the sign determination system according to the present embodiment. Here, for example, a case where the determination device is implemented by a vehicle 10 will be described. As illustrated in FIG. 2, the vehicle 10 includes a control section 100, a communication section 110, a sensor section 120, a head swing section 130, and a storage section 140.

(Control Section 100)

The control section 100 functions as a computational processing device or a control device, and controls partial or overall operations of respective structural elements on the basis of various kinds of programs recorded on a ROM, a RAM, the storage section 140, a removable recording medium, or the like.

The control section 100 according to the present embodiment may also function as a sign determination section 101 and a head swing control section 102. The sign determination section 101 determines a sign related to a decrease in driving ability of a driver. Specifically, the sign determination section 101 has a function of determining the sign related to a decrease in driving ability of the driver on the basis of head movement measured by a head movement measurement section 121 and eye movement measured by an eye movement measurement section 122. More specifically, the sign determination section 101 may particularly determine a sign related to sleepiness on the basis of speed of the eye movement and speed of the head movement. The sign determination section 101 continuously determines the signs, and takes various kinds of measures in the case where it is determined that the movements show the sign of a decrease in the driving ability.

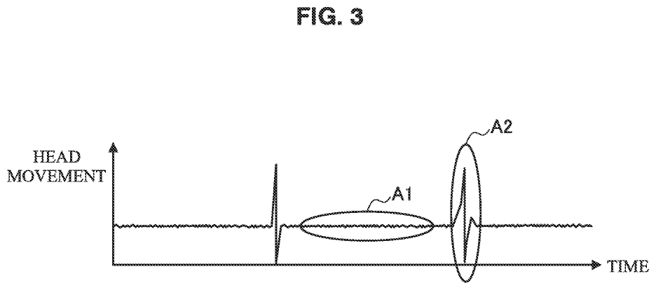

The head swing control section 102 performs control to operate the head swing section 130 in such a manner as to swing the head of the driver. As described above, the VOR is not induced if the head does not move, and it is not possible to determine the sign. Therefore, the head swing control section 102 operates the head swing section 130 to move the head and induce the VOR. Here, FIG. 3 illustrates an example of measurement data of the head movement. With reference to the measurement data (for example, acceleration data) of the head movement illustrated in FIG. 3, data A1 does not include the head movement. Therefore, the VOR is not induced and it is not possible to determine the sign. However, data A2 includes the head movement because the head swing control section 102 performs the control to operate the head swing section 130. Therefore, magnitude of the head movement is desirably large enough to induce the vestibulo-ocular reflex in the case of determining the sign on the basis of the vestibulo-ocular reflex. Operation intensity (parameter) of the head swing section 130 for causing the head movement that is large enough to induce the vestibulo-ocular reflex may be newly set or may be personalized. In the case of personalization, for example, the head swing control section 102 may operate the head swing section 130 at a time when it is highly possible that the driver is in an arousal state such as the time when the driver starts driving, and the head swing control section 102 may record degrees of the operation intensity that has induced the vestibulo-ocular reflex. In addition, the head swing control section 102 may set the operation intensity (parameter) depending on physical features (height, weight, sitting height, and the like) of the driver.

Note that, depending on intensity of the head swing, it is also assumed that the driver notices the head swing in the arousal state. However, a bit of swing is expected to some extent during driving, and it is conceivable that the driver does not feel annoyance or unnaturalness (the annoyance and unnaturalness are acceptable). However, in consideration of ride comfort to the driver, the head swing control section 102 may perform control in such a manner as to cause the head movement that is large enough to induce the vestibulo-ocular reflex but does not deteriorate the ride comfort. For example, when controlling the head swing section 130, the head swing control section 102 may treat amplitude and frequency of vibration as a control target. The head swing control section 102 is capable of adjusting the amplitude and frequency of vibration and performing control in such a manner as to cause the head movement that is large enough to induce the vestibulo-ocular reflex but does not deteriorate the ride comfort.

In addition, for example a timing of the head swing control performed by the head swing control section 102 may be as follows.

The head swing control section 102 may control the head swing at certain intervals. This makes it possible to periodically induce the vestibulo-ocular reflex and improve the accuracy of determining the sign. In addition, the “certain interval” is variable. The head swing control section 102 is capable of changing the timing of the head swing control from every five minutes to every minute or the like, depending on a situation (such as a situation of the driver, an environment such as a driving condition or time, a situation of traffic around the driver). In addition, the head swing control section 102 may control the swing in the case where the head movement is smaller than a predetermined value. The “predetermined value” is assumed to represent magnitude of the head movement that induces the VOR, for example. This makes it possible to control head swing as necessary and improve the accuracy of determining the sign.

In addition, the head swing control section 102 may control the head swing in the case where the VOR has not been detected for a predetermined period of time or the head has not moved for the predetermined period of time (or the head movement is smaller than the predetermined value). The “predetermined period of time” is variable. The head swing control section 102 is capable of changing the predetermined period of time depending on a situation (such as a situation of the driver, an environment such as a driving condition or time, a situation of traffic around the driver). In addition, the “predetermined value” is assumed to represent magnitude of the head movement that induces the VOR, for example. The predetermined value may be newly set or may be personalized. It is assumed that the driver may notice the head swing control in the case where the head swing control is performed while the driver is in the arousal state. However, it is possible to reduce the number of times of the head swing control and improve the accuracy of determining the sign by performing the head swing control in the case where the VOR has not been detected or the head has not moved for a predetermined period of time.

In addition, the head swing control section 102 may control the head swing at a timing depending on a state of the mobile object. For example, the timing depending on the state of the mobile object is a timing when the mobile object is assumed to vibrate. Specifically, it may be assumed that the mobile object is vibrated by change in operation input to a steering wheel, a brake pedal, or an accelerator pedal (internal factor), or an ambient environment such as an intersection or a bump on a road (external factor). Therefore, it is possible to more certainly prevent the driver from feeling unnatural head swing by controlling the head swing in a situation where the mobile object is assumed to shake to some extent.

In addition, the head swing control section 102 may control the head swing at a timing depending on a state of the driver. For example, the head swing control section 102 may control the head swing at a timing when driving ability of the driver is predicted to decrease. The timing when the driving ability of the driver is predicted to decrease may be newly set or may be personalized through learning. Specifically, time elapsed after the start of driving, biological information of the driver (pulse rate, heart rate, body temperature, respiration, etc.), a degree of fatigue, a driving situation (a situation where the vehicle 10 has gone straight continuously, a situation where the steering wheel has slightly operated, a situation where the driver has continuously repressed the accelerator pedal, etc.), a time zone, temperature, humidity, and the like may be assumed.

In addition, in the case where a plurality of the head swing sections 130 is installed, the head swing control section 102 may randomly decide which of the head swing sections 130 is to be controlled, may control the plurality of head swing sections 130 in a predetermined order, may simultaneously control the plurality of head swing sections 130, may decide which of the head swing sections 130 is to be controlled depending on the driving situation or the like, or may personalize each of the head swing sections 130 (for example, by measuring likelihood of inducing the driver's VOR when the driver starts driving).

(Communication Section 110)

The communication section 110 is a communication device for transmitting and receiving data to and from an outside, and can be connected to the Internet by, a wired or wireless LAN, Bluetooth (registered trademark), Wi-Fi (registered trademark), a low-power wide-area (LPWA) network, or the like, for example. In addition, for example, the communication section 110 may be implemented by a data communication module (DCM) and perform communication in conformity with a communication standard such as LTE or the like.

For example, the communication section 110 according to the present embodiment may receive various kinds of parameters to be used for determining the sign and controlling the head swing from a predetermined server, or may transmit a sign determination result to the outside (such as another vehicle or a predetermined server on a network).

(Sensor Section 120)

The sensor section 120 includes the head movement measurement section 121 and the eye movement measurement section 122, and measures head movement and eye movement. The head movement and the eye movement may be measured by a single sensor (such as the camera 120a) or may be measured by different sensors. In addition, the sensor section 120 may be a contact sensor section or a non-contact sensor section.

For example, the head movement measurement section 121 may performs image processing on the captured images of the head of the driver, and may calculates an amount of change in position of the head between the images. Alternatively, the head movement measurement section 121 may detect movement of the head of the driver by a ranging sensor using infrared light, a microwave, a Doppler wave, or the like.

In addition, the eye movement measurement section 122 may measure movement of the eyes of the driver on the basis of the images of the eyeballs captured by the light receptive section, for example. Alternatively, the eye movement measurement section 122 may measure movement of the eyeballs of the driver on the basis of ocular potential detected by the ocular potential sensor.

(Head Swing Section 130)

The head swing section 130 is a mechanism configured to swing the head of the driver. For example, the head swing section 130 may be a webbing winding device configured to wind the webbing worn by the driver, an adjustment mechanism of the driver's seat, or a vibration section installed in the driver's seat. For example, it is possible to apply tension to the webbing and cause the head movement by temporarily winding the webbing. Alternatively, it is also possible to cause the head movement by temporarily moving a sitting surface of the driver's seat, a reclining seat back, or a head restraint in an up-down direction or a front-rear direction (sliding direction). In addition, it is also possible to install the vibration section in the seat back, the head restraint, or the like and causes the head movement through vibration.

(Storage Section 140)

The storage section 140 is a device configured to store various kinds of data. The storage section 140 is, for example, a magnetic storage device such as a hard disk drive (HDD), a semiconductor storage device, an optical storage device, a magneto-optical storage device, or the like.

The configuration example in which the determination device according to the present embodiment is implemented by the vehicle 10 has been described above. However, the configuration according to the present embodiment is not limited to the example illustrated in FIG. 2. For example, the determination device according to the present embodiment may be implemented by a device that is separate from the vehicle, and may have the functions of the control section 100 illustrated in FIG. 2. Specifically, the determination device according to the present embodiment may be implemented by a smartphone, a tablet terminal, or the like carried by the driver. In this case, the smartphone, the tablet terminal, or the like connects to an in-vehicle network, receives head swing movement measurement data and eye movement measurement data from the vehicle, determines the sign (by the sign determination section), and transmits a head swing control signal to the vehicle (from the head swing control section).

<3. Operation Process>

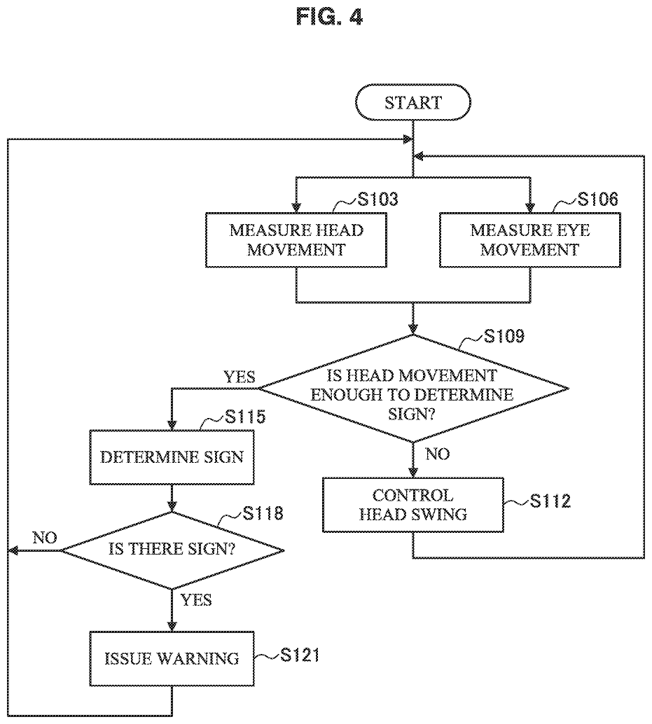

FIG. 4 is a flowchart illustrating an example of a flow of a sign determination process performed by the determination device according to the present embodiment. Such a process may start when an engine starts or when the driver sits on the driver's seat.

As illustrated in FIG. 4, the sensor section 120 first measures head movement by using the head movement measurement section 121 (Step S103), and measures the eye movement by using the eye movement measurement section 122 (Step S106). Such measurement may be performed continuously, periodically, irregularly, or at a predetermined timing.

Next, the sign determination section 101 judges whether or not head movement measurement data is enough to determine the sign (Step S109). Specifically, for example, the sign determination section 101 judges whether or not head movement enough to induce the VOR has been measured. A threshold of the head movement enough to induce the VOR may be set in advance, or may be set for each driver. In addition, the threshold may include a lower limit and an upper limit.

Next, in the case where the head movement is not enough (NO in Step S109), the head swing control section 102 controls the head swing section 130 in such a manner that the head swing section 130 swings the head (Step S112). This makes it possible to induce the VOR, and it is expected to determine the sign. Note that, the flowchart illustrates the case where the head is swung in the state where the head movement is not enough, as an example. However, as described above, many examples are assumed with regard to the timing of swinging the head.

On the other hand, in the case where the head movement is enough (YES in Step S109), the sign determination section 101 detects the VOR on the basis of the head movement measurement data and the eye movement measurement data and determines a sign of a decrease in the driving ability (Step S115).

Next, in the case where it is determined that there is the sign of the decrease in the driving ability (YES in Step S118), the control section 100 issues a warning (Step S121) in a process of awaking the driver. The warning may be issued by sound, for example.

Alternatively, in the case where there is no sign (NO in Step S118), the process returns to initial processes (processes in Step S103 and Step S106) in the flowchart.

The above-described Step S103 to Step S121 are continuously repeated until a predetermined termination condition is satisfied, for example, until the engine stops or until the driver is away from the driver's seat.

<4. Supplement>

The case where the vehicle is used as an example of the mobile object has been mainly described above. However, the mobile object according to the present embodiment is not limited to the vehicle, and may be a ship, an airplane, or the like. In addition, the sign determination system according to the present embodiment is not limited to the determination of the sign of the driver who actually rides in and drives the mobile object. It is also possible to determine a sign of a driver who remotely drives the mobile object, or a driver who does driving simulation.

In addition, the sign determination according to the present embodiment may be determination of a sign related to various events that is factors of a decrease in driving ability such as sleepiness, an attack of chronic disease like epilepsy, fainting caused by anemia, or a cerebral infarction.

For example, it is known that, in the case where the driver has an epileptic seizure and a frontal eye field is a symptomatogenic zone, a versive seizure occurs and the head and eyes turns in a direction opposite to a focus of disease. In addition, sometimes characteristic eye movement such as rotation may occur in the case of fainting caused by anemia. In the case of cerebral infarction, sometimes one of the eyes may become paralyzed.

Therefore, the sign determination section 101 according to the present embodiment is also capable of determining signs other than the sign related to sleepiness by comparing characteristic behavior of measured eye movement and head movement with characteristic behavior of eye movement and head movement related to the above-described events.

<5 Conclusion>

Although details of the preferable embodiment of the present invention has been described above with reference to the appended drawings, the present invention is not limited thereto. It will be clear to a person of ordinary skill in the art of the present invention that various modifications and improvements may be obtained within the scope of the technical idea recited by the scope of the appended claims, and it should be understood that they will naturally come under the technical scope of the present invention.

In addition, the effects described herein are illustrative or exemplary but not limitative. That is, besides the above effects or instead of the above effects, the technology according to the present disclosure may provide other effects that are obvious to a person skilled in the art.

In addition, the processes in the respective steps related to the determination device in the present specification are not necessarily performed in time sequence in the order illustrated in the flowchart. For example, the processes in the respective steps related to the determination device may be performed in a different order from the order illustrated in the flowchart, or may be processed in parallel.

In addition, a program for causing hardware such as a CPU, a ROM, or a RAM installed in a computer to exert functions equal to those of the structural elements of the determination device can be created. Also, a computer readable recording medium having the program recorded therein may be provided.

### REFERENCE SIGNS LIST

10 vehicle
100 control section
101 sign determination section
102 head swing control section
110 communication section
120 sensor section
121 head movement measurement section
122 eye movement measurement section
130 head swing section
140 storage section

1. A determination device comprising:
a sign determination section configured to determine a sign related to a decrease in driving ability of a driver who drives a mobile object on a basis of head movement measurement data obtained by measuring head movement of the driver and eye movement measurement data obtained by measuring eye movement of the driver; and
a head swing control section configured to control a head swing section in such a manner that the head swing section swings the head of the driver.

2. The determination device according to claim 1,
wherein the head swing control section controls the head swing section at a timing depending on at least a state of the mobile object or a state of the driver.

3. The determination device according to claim 2,
wherein the timing depending on the state of the mobile object is a timing when the mobile object is assumed to vibrate.

4. The determination device according to claim 2,
wherein the timing depending on the state of the driver is a timing when the driving ability of the driver is predicted to decrease.

5. The determination device according to claim 1,
wherein the head swing control section controls the head swing section in a case where the head movement measurement data is smaller than a predetermined value.

6. The determination device according to claim 5, wherein
the sign determination section detects vestibulo-ocular reflex induced by the head movement on a basis of the head movement measurement data and the eye movement measurement data, and determines the sign related to the decrease in the driving ability of the driver on a basis of the vestibulo-ocular reflex, and
the predetermined value is a value of the head movement that makes it possible to induce the vestibulo-ocular reflex.

7. The determination device according to claim 5,
wherein the head swing control section controls the head swing section in a case where head movement beyond the predetermined value has not been measured for a predetermined period of time.

8. The determination device according to claim 1,
wherein the head swing control section controls the head swing section at a certain interval.

9. The determination device according to claim 1,
wherein the head swing section is a webbing winding device configured to wind webbing worn by the driver, an adjustment mechanism of a driver's seat, or a vibration section installed in the driver's seat.

10. The determination device according to claim 9,
wherein the head swing control section controls at least any of the webbing winding device, the adjustment mechanism of the driver's seat, or the vibration section installed in the driver's seat.

11. A non-transitory computer readable recording medium storing a program that causes a computer to function as:
a sign determination section configured to determine a sign related to a decrease in driving ability of a driver who drives a mobile object on a basis of head movement measurement data obtained by measuring head movement of the driver and eye movement measurement data obtained by measuring eye movement of the driver; and
a head swing control section configured to control a head swing section in such a manner that the head swing section swings the head of the driver.
